(19) United States
(12) Patent Application Publication
(10) Pub. No.: US 2009/0192600 A1
(43) Pub. Date: Jul. 30, 2009

(54) Sizing device having two sizers and methods of use

(76) Inventor: Timothy R. Ryan, Shorewood, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 12/315,950

(22) Filed: Dec. 8, 2008

Related U.S. Application Data
(60) Provisional application No. 61/062,477, filed on Jan. 25, 2008.

(51) Int. Cl. A61F 2/24

(52) U.S. Cl. 623/2.11

(57) ABSTRACT

A sizing device for measuring a valve annulus, said sizing device comprising: an elongate segment having first and second ends; a first sizer attached to the first end of the elongate segment; and a second sizer attached to the second end of the elongate segment, wherein the first and second sizers have different shapes that correspond to the same size of valve annulus. A method of measuring a valve annulus of a heart. A system for measuring a valve annulus of a heart.

### PRIORITY

The present non-provisional patent application claims benefit from United States Provisional Patent Application having Ser. No. 61/062,477, filed on Jan. 25, 2008, by Timothy R. Ryan, and titled SIZING DEVICE HAVING TWO SIZERS AND METHODS OF USE, wherein the entirety of said provisional patent application is incorporated herein by reference.

### TECHNICAL FIELD

The present invention relates generally to devices and methods for repair of heart valves. More particularly, the present invention relates to a device including one or two sizers with shapes corresponding to valve annuli and/or annuloplasty devices, which is used to measure the size of a heart valve annulus during surgery, in order to select a properly sized valve or annuloplasty ring or band for replacement or repair of a defective heart valve. However, the sizer could also apply for other types of valves such as venous valves.

### BACKGROUND OF THE INVENTION

Heart valve disease is a widespread condition in which one or more of the valves of the heart fails to function properly. Various surgical techniques may be used to replace or repair a diseased or damaged valve. Damaged leaflets of the valve may be excised and the annulus sculpted to receive a replacement valve. Another less drastic method for treating defective valves is repair or reconstruction by annuloplasty, in which the effective size of the valve annulus is contracted and reinforced, by attaching a prosthetic annuloplasty ring or band to an interior wall of the heart around the valve annulus. The annuloplasty ring or band is designed to support the functional changes that occur during the cardiac cycle, while maintaining leaflet coaptation and valve integrity.

To perform successful valve replacement and annuloplasty surgery, the size of the valve annulus must be accurately measured. Sizing may be achieved by measuring the width and the height of the anterior leaflet of the mitral valve, for example, using sizing obturators. Another way to size the annulus is to use valve sizers, which resemble the shape of the valve annulus and are provided in various sizes. In order to use valve sizers, a surgeon estimates the native valve annulus size and selects a sizer accordingly. The sizer is attached to the end of the handle and guided into proximity of the annulus. If the sizer is not the appropriate size, it is withdrawn, detached from the handle, and replaced by a different sizer. Once the size of the annulus has been determined, a properly sized valve or annuloplasty ring or band may be selected and implanted.

Although numerous configurations of valve annulus sizers are currently in use, there remains a need for improved sizing devices and related methods in order to improve the efficiency of and results associated with valve repair or replacement surgery.

### SUMMARY OF THE INVENTION

The present invention relates to a valve sizing device comprising sizer with three-dimensional (3D) profile or shape that matches an annuloplasty device or is shaped to match a native, mitral valve annulus. The sizer heads may be snapped or threaded or permanently attached on a handle or delivery device. Preferably the sizing device includes two sizers with different shapes corresponding to the same size of replacement heart valve or annuloplasty device, that is used to measure the size of a heart valve annulus during surgery, in order to select a properly sized valve or annuloplasty device for replacement or repair of a defective heart valve. However, the present invention also more generally relates to a device that includes one or two sizers that have a two-dimensional (2D) and/or 3D shape that matches the shape of a native mitral valve annulus at a portion of the cardiac cycle, or that matches the actual shape of a saddle-shaped annuloplasty device. Additionally, the valve sizing device may be used for valves other than heart valves, with venous valves being an example.

Embodiments of the present invention offer numerous advantages. One advantage is that the native mitral valve annulus assumes various 2D and 3D shapes throughout the cardiac cycle. The ability of a surgeon to compare a diseased or repaired annulus with a 3D sizer having a shape matching that of a normal, healthy valve annulus at a portion in the cardiac cycle will enable improved sizing and improved valve repair. Another advantage is that having two differently shaped sizers on one device that correspond to a single valve or annuloplasty device size, provides the surgeon with an ability to better choose the appropriateness of a proposed size and shape of annuloplasty device for an annulus. The ability to verify the native or repaired annulus size with more than one sizer shape or configuration may result in a better fit and result for a chosen annuloplasty device, for example. Another advantage is that having differently shaped sizers on the same device that correspond to a certain size of annuloplasty device provides a more efficient way for a surgeon to verify the size of the annulus, since the surgeon does not have to use two separate sizers. The surgeon merely has to switch ends of one sizer in order to size an annulus with two differently shaped sizers corresponding to one size of annuloplasty device. Thus, the surgeon does not have to attach and detach different sizers on the end of a sizer device handle.

A first aspect of the present invention is a sizing device for measuring a valve annulus. One embodiment of the sizing device comprises: an elongate segment having first and second ends; a sizer attached to the first end of the elongate segment, wherein the sizer is shaped to match a native mitral valve annulus at a portion of a cardiac cycle and/or is shaped to match a saddle shape of a corresponding annuloplasty device.

A second embodiment of the sizing device comprises: an elongate segment having first and second ends; a first sizer attached to the first end of the elongate segment; and a second sizer attached to the second end of the elongate segment, wherein the first and second sizers have different shapes that correspond to the same size of valve annulus. The first and second sizers may differ in two-dimensional and/or three-dimensional shape. The first sizer may be planar in shape and the second sizer may have three-dimensional shape. The first and second sizers may include opposing notches located on opposite sides of the sizers, wherein the opposing notches are provided to facilitate viewing of portions of the valve annulus upon placement of the sizers into close proximity to the valve annulus.

A second aspect of the present invention is a method of measuring a valve annulus of a heart. One embodiment of the method comprises the steps of: providing a plurality of sizing devices, each sizing device comprising: an elongate segment having first and second ends; a first sizer attached to the first end of the elongate segment; and a second sizer attached to the second end of the elongate segment; wherein the first and second sizers have different shapes that correspond to the same size of valve annulus; selecting one of the plurality of sizing devices; positioning the first sizer of the one sizing device on the valve annulus; and turning the one sizing device such that the first sizer is moved off the valve annulus and such that the second sizer is positioned on the valve annulus. The method may further comprise the step of: repeating the selecting, positioning and turning steps until the size of the valve annulus is determined. The plurality of sizing devices may correspond to a plurality of annuloplasty devices, and the method may further comprise the steps of: providing a plurality of annuloplasty devices; and selecting one of said annuloplasty devices which has the size that corresponds to the determined size of the valve annulus. The method may further comprise the step of: implanting the selected one annuloplasty device on the valve annulus.

A third aspect of the present invention is a system for measuring a valve annulus of a heart. One embodiment of the system comprises: a plurality of sizing devices each sizing device comprising: an elongate segment having first and second ends; a first sizer attached to the first end of the elongate segment; and a second sizer attached to the second end of the elongate segment; wherein the first and second sizers have different shapes that correspond to the same size of valve annulus; wherein each of the plurality of sizing devices corresponds to one of a plurality of valve annulus sizes that also corresponds to one of a plurality of annuloplasty devices.

Other aspects, features and advantages of the present invention will become apparent to those persons having ordinary skill in the art to which the present invention pertains from the following description taken in conjunction with the accompanying drawings.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of an exemplary sizing device in accordance with the present invention;

FIG. 2 is a side view of the exemplary sizing device shown in FIG. 1;

FIG. 3 is a close-up, perspective view of the first sizer shown on the sizing device in FIG. 1;

FIG. 4 is a bottom planar view of the first sizer shown in FIG. 3;

FIG. 5 is a close-up, perspective view of the second sizer shown on the sizing device in FIG. 1;

FIG. 6 is a bottom planar view of the second sizer shown in FIG. 5;

FIG. 7 is a perspective view of another exemplary sizing device in accordance with the present invention; and

FIG. 8 is a side view of the exemplary sizing device shown in FIG. 7.

### DETAILED DESCRIPTION OF PREFERRED EMBODIMENTS

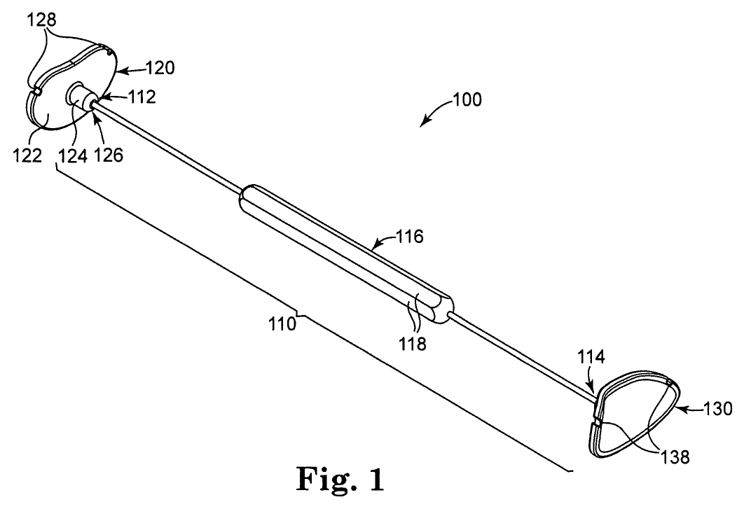

With reference to the accompanying figures, wherein like components are labeled with like numerals throughout the several figures, and, initially, to FIG. 1, one embodiment of one aspect of the present invention is shown. FIG. 1 shows a perspective view of a sizing device 100 comprising an elongate segment 110 with first 120 and second 130 valve sizers on first 112 and second 114 ends of the elongate segment 110. The elongate segment 110 also includes a handle 116 that may optionally be provided in order for the sizing device 110 to be handled more easily. The handle 116 is shown with optional bevels 118 on the outer surface, in order to allow for better gripping of the device 100 by a user.

The elongate segment 110 may be all one piece of material, such as a metal wire that runs through the handle 116. Alternatively, the elongate segment 110 may comprise two pieces of metal wire, for example, that are secured in the handle 116. The present invention is not limited to the use of metal wire for the elongate segment 110, however. Other materials are also contemplated.

The handle 116 is preferably comprised of a polymeric material. However other materials are also contemplated.

The first 120 and second 130 valve sizers, shown on opposite ends of the elongate segment 110 preferably have different shapes. The first valve sizer 120 is substantially flat in shape relative and normal to the elongate segment 110, but includes 2D shape. The second valve sizer 130 has 2D shape and also is curved in shape relative and normal to the elongate segment 110, which provides 3D shape. The 2D and 3D shapes of first and second valve sizers 120, 130 are exemplary, and others are contemplated by the present invention.

Although the shapes of the first 120 and second 130 sizers on the sizing device 100 are preferably different, the sizers 120, 130 preferably correspond to the same size of valve annulus. The size of valve annulus is generally determined by either measuring the major diameter of the annulus, the distance between left and right trigones, and/or the distance between anterior and posterior commissures. Mitral annuloplasty devices are generally available starting with a size of 24 mm and increasing by 2 mm each. Therefore, the first and second sizers 120, 130 preferably have a size ranging from about 24 mm to about 40 mm, and at 2 mm increments. However, it is contemplated that the sizers 120, 130 may have additional sizes.

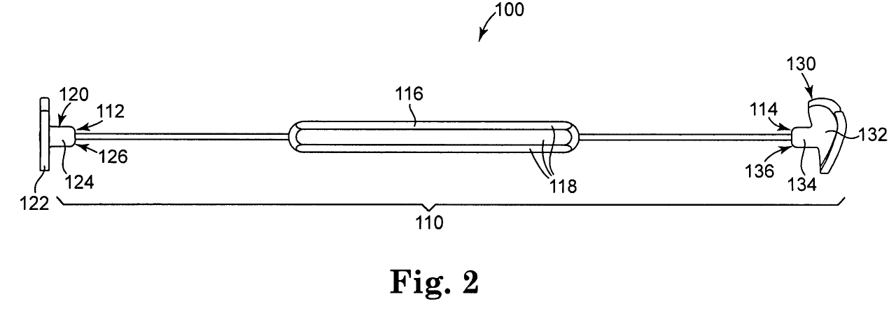

FIG. 2 shows a side view of the sizing device 100 of FIG. 1. The differences in the 3D shapes of the first 120 and second 130 valve sizers is shown in the figure.

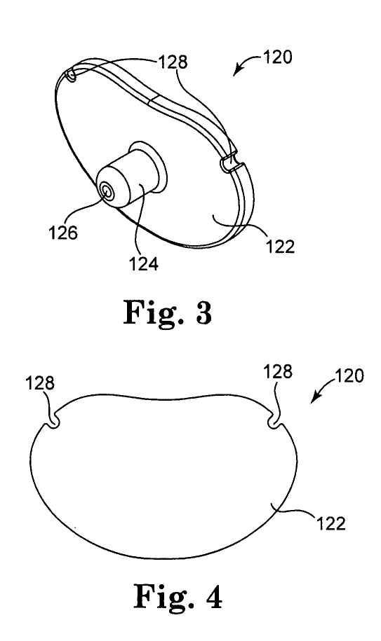

FIG. 3 shows a close-up, perspective view of the first valve sizer 120 of FIG. 1. Detail of the first valve sizer 120 is shown, which is that the valve sizer 120 comprises a sizing plate 122 and an attachment hub 124. The attachment hub 124 includes a socket 126 into which the elongate segment 110 of the device 100 extends and is secured. In the embodiment shown in FIGS. 1 and 2, the elongate segment 110 is permanently adhered or secured to the first 120 and second 130 valve sizers. However, it is also contemplated that the valve sizers 120, 130 may be configured such that they are releasably attachable to the elongate segment 110. For example, the sizers 120, 130 may be snap-fit onto the elongate segment 110 or threaded onto the elongate segment 110, or any combination of any such attachment means.

FIG. 3 also shows notches 128 in the sizing plate 122. These notches are located in sizing plate 122 as shown, such that the sizing device 100 is designed for sizing a mitral valve annulus. The present invention is not, however, limited to application to the mitral valve annulus. The notches 128 are provided for the purpose of being able to visibly identify the position of anterior and posterior trigones of a mitral valve annulus. The trigones will preferably be seen in both notches 128 if the size of the sizing device is proper. Alternatively, the notches 128 may be provided for the purpose of being able to identify the position of anterior and posterior commissures of a mitral valve annulus for the purpose of sizing the annulus. The mitral valve is suspended from the fibrous skeleton of the heart and particularly from both the anterior (left) and posterior (right) trigones. The notches 128 allow for direct and un-obscured viewing of the left and right trigone/commissures positions while the sizing plate 122 is inserted in the mitral valve annulus. Further, the notches 128 establish the precise location for suture points if an annuloplasty device is ultimately employed in the annulus.

It should be noted that the notches 128 could alternatively be replaced by markings or other distinguishing features. It is also contemplated that the sizing plate 122 of the present invention may either include no notches, a different number of notches, or notches in different locations than those shown, depending upon the anatomy of the particular valve being repaired.

FIG. 4 shows an end view of the sizing device 100 from the end of the device including the first valve sizer 120, and provides a bottom planar view of the first valve sizer 120. The figure shows the 2D shape of the sizer 120, including notches 128. The 2D shape of the sizer 120 generally resembles the shape of a mitral valve in a flaccid configuration, such as when the heart is on bypass during annuloplasty surgery. Other sizers having different 2D shapes are also contemplated by the present invention.

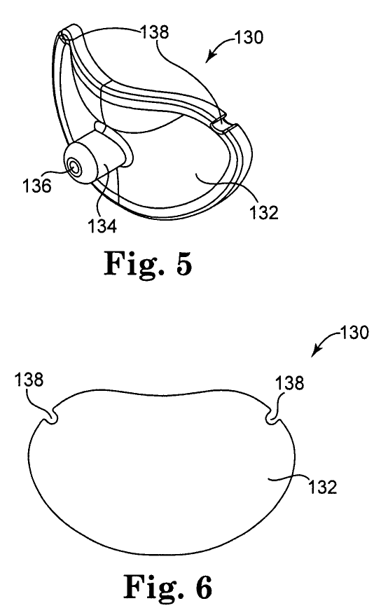

FIG. 5 shows a close-up, perspective view of the second valve sizer 130 of FIG. 1. Detail of the second valve sizer 130 is shown, which is that the valve sizer 130 comprises a curved sizing plate 132 and an attachment hub 134. The curved, or 3D, shape of the sizing plate 132 is not present in the first valve sizer 120, which is planar. The 3D shape of the sizing plate 132 of the sizer 130 may resemble the shape of a native mitral valve annulus during some portion of a cardiac cycle, or the saddle shape of an annuloplasty device. Other 3D shapes, beside those shown, are also contemplated by the present invention.

The attachment hub 134 on sizer 130 includes a socket 136 in which the second end 114 of the elongate segment 110 of the device 100 is retained. The sizing plate 132 includes notches 138. The attachment hub 134 and notches 138 are similar to those in configuration and function as the hub 124 and notches 128 in the first sizer 120 and the discussion above also applies to these features.

FIG. 6 shows an end view of the sizing device 100 from the end including the second valve sizer 130. The view provides a bottom planar view of the second valve sizer 130. The bottom planar view shows the 2D shape of the sizer 130, including notches 138. The 2D shape shown in FIG. 6 is substantially similar to that of the first valve sizer 120 shown in FIG. 4. The 2D shapes of the two sizers in the present invention may be substantially similar in some embodiments. However, in other embodiments, the 2D shapes may be different.

In sizing device 100, the purpose of having two different shapes of sizers corresponding to the same size of valve annulus may be to allow the size of the annulus to be measured while having different shapes, such as that may exist during different portions of a cardiac cycle, for example. Alternatively, the use of two different shapes of sizers may be to allow a surgeon to check to see what shape of annuloplasty device, for example, may best fit in a valve annulus of a given size. In the case of sizing device 100, the surgeon could use the device 100 to check if a planar ring or a ring having 3D shape would be best suited for a particular valve annulus of a given size.

The sizers, and the sizing plate of the sizers, in particular, may have one of a plurality of possible 2D and 3D shapes. The shape of the sizers depends upon, e.g., the type of valve being sized, the disease state of the valve, the shape of a corresponding annuloplasty device, etc. Thus, it is contemplated that the sizing devices of the present invention include a combination of two sizers, with each sizer having one of a plurality of possible shapes. The combinations of shapes of sizers shown in the accompanying figures are exemplary, and other possibilities and combinations are also contemplated.

The sizers (e.g., sizers 120 and 130) of the present invention are preferably made from biocompatible material that is also preferably optically transparent and rigid to the degree that it maintains a shape. The material could have a degree of deformability to minimize tissue trauma while introducing the sizer through the surgical incision site. An exemplary material for the sizers is polysulfone or another similar thermoplastic. However, other materials are also contemplated.

The thickness of the sizing plate (e.g., sizing plates 122 and 132) of the sizer is preferably minimized while still retaining substantial strength to prevent substantial flexing or bending or to prevent breakage. The thickness is minimized in order to prevent optical distortion through the sizing plate and/or in order to allow the sizing plate to fit through relatively small openings, such as an annulus. The sizing plate is shown as a continuous surface. However, such continuous sizers could be replaced by spokes terminating at a central hub, for example. Another configuration of the sizer could be to have an outer periphery of the sizer that matches the profile and geometry of a corresponding annuloplasty device. Remaining geometry of the sizer could be minimized to allow the surgeon maximal visualization of the implant prior to implanting the actual device, for example.

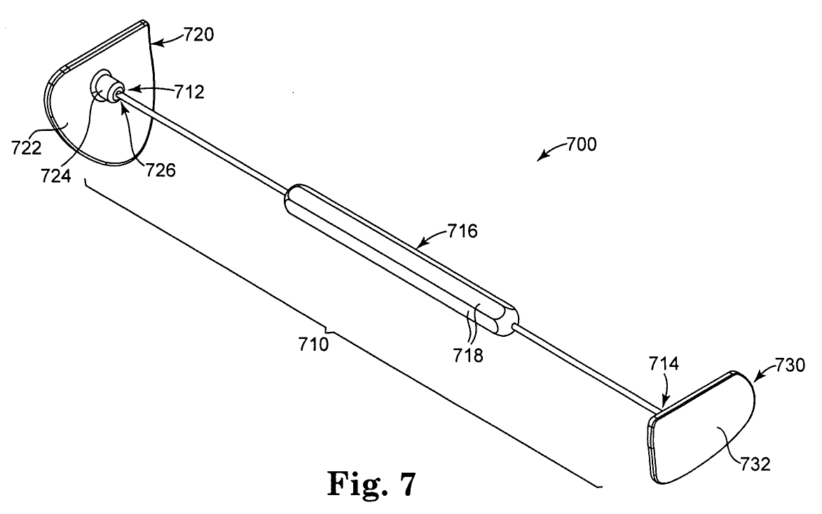

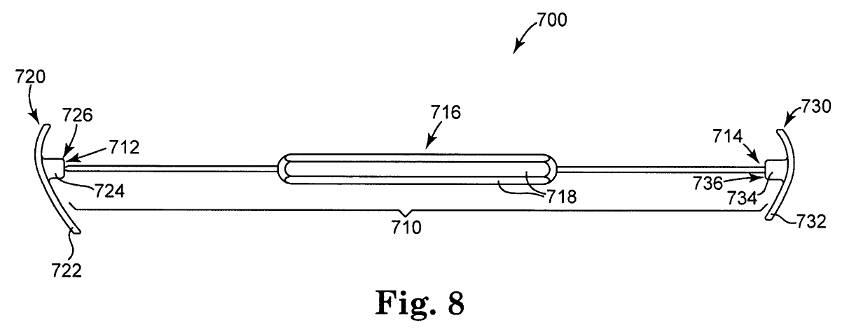

FIGS. 7 and 8 show perspective and side views, respectively, of another embodiment of the sizing device of the present invention. Sizing device 700 includes an elongate segment 710, similar to that in sizing device 110 (FIG. 1), including an optional handle 716 with optional bevels 718 on the outer surface to provide the user with a better grip on the device 700. The sizing device 700 also includes first 720 and second 730 sizers on opposite ends 712, 714 of the elongate segment 710. First 720 and second 730 valve sizers include sizing plates 722, 732 that are used to size a valve annulus. First 720 and second 730 valve sizers also include attachment hubs 724, 734 that include sockets 726, 736 in which the elongate segment 710 is retained. Optionally, first 720 and second 730 valve sizers are releasably attached to the elongate segment 710.

The shapes of first 720 and second 730 valve sizers are different from each other in FIGS. 7 and 8. The shapes are also different from those shown in sizing device 110 (FIG. 1). The two different shapes of sizers 720, 730 on sizing device 700 preferably correspond to the same size valve annulus (i.e., have the same length along the major-axis), and also substantially match the shapes or possible shapes of two annuloplasty devices that may be implanted in the annulus. The shapes of sizers 720, 730 included in the embodiments shown are exemplary, however, and other shapes are also contemplated.

Preferably, first and second valve sizers on each sizing device having different shapes, but correspond to the same size of valve annulus. In order to measure the size of a valve annulus accurately, a plurality of sizing devices corresponding to a plurality of valve annulus sizes are provided and tested in the annulus by a surgeon. As known in the art, sizers are numbered according to the major-axis length in millimeters. The numbering system for mitral annulus sizers, for example, includes 24, 26, 28 . . . 40.

Another embodiment of the present invention comprises one sizer on the sizing device. The one sizer is shaped to match a native mitral valve annulus at a portion of a cardiac cycle and/or is shaped to match a saddle shape of a corresponding annuloplasty device.

A second aspect of the present invention relates to methods of using the sizing devices described above. One embodiment is a method of measuring a valve annulus of a heart. Although the sizing device of the present invention may be used in any applicable type of procedure in which it is necessary to measure a valve annulus, the present invention is particularly useful in annuloplasty surgery. The method, as used during annuloplasty surgery, comprises the steps of: providing a plurality of sizing devices of the present invention, and as described above; selecting one of the plurality of sizing devices; positioning the first sizer of the one sizing device on the valve annulus; and turning the one sizing device such that the first sizer is moved off the valve annulus and such that the second sizer is positioned on the valve annulus. The surgeon is able to use the same hand holding the sizing device in order to turn the sizing device to remove the first sizer from the annulus and position the second sizer in the annulus. Therefore, the surgeon only needs one hand to test the size of the annulus using two sizers. The method may further comprise the step of: repeating the steps of the method using other sizing devices in the plurality of sizing devices as necessary until the size of the valve annulus is determined.

The sizing device, as described above, preferably includes two sizers having different 2D and/or 3D shapes. Therefore, the method may include the step of determining and selecting the desired shape of sizer that best fits in a valve annulus. Annuloplasty devices are preferably available having corresponding 2D and/or 3D shapes.

During annuloplasty surgery, for example, a plurality of annuloplasty rings or bands (devices), each having the same shape generally, but of a different size as known in the art, are provided. A plurality of sizing devices of the present invention are also made available to the surgeon. Each of the sizing devices has a size and possibly a shape corresponding to one of the annuloplasty devices. The method may then further comprise the steps of: providing a plurality of annuloplasty devices each of different size; and selecting one of said annuloplasty devices which has a size and/or shape corresponding to that of the valve annulus. The method may then further comprise the step of: implanting the selected annuloplasty device on the valve annulus.

Another embodiment of the present inventive method is a method of measuring a valve annulus of a heart, the method comprising the steps of: providing a plurality of sizing devices, each sizing device comprising: an elongate segment having first and second ends; a sizer attached to the first end of the elongate segment; wherein the sizer is shaped to match a native mitral valve annulus at a portion of a cardiac cycle; selecting one of the plurality of sizing devices; and positioning the sizer of the one sizing device on the valve annulus. Alternatively, the sizer may be shaped to match a saddle shape of a corresponding annuloplasty device.

A third aspect of the present invention is a system for measuring a valve annulus of a heart, the system comprising: a plurality of sizing devices of the present invention, wherein each of the plurality of sizing devices corresponds to one of a plurality of valve annulus sizes.

It is to be understood that while particular embodiments of the invention have been illustrated for use in typical valve repair procedures, various modifications to shape, and arrangement of parts can be made as may be desirable for varying applications as may relate to valve sizes or later developed techniques. The invention should not be considered limited to the specific methods and devices precisely described herein. On the contrary, various modifications will be apparent to those of ordinary skill upon reading the disclosure. Although certain embodiments are described with reference to the mitral valve, use with other valves or anatomical structures is also contemplated. Additionally, the sizing devices according to the invention can be made of disposable materials, for one-time use, or of non-disposable materials, for re-sterilization and subsequent reuse. The foregoing detailed description has been given for clarity of understanding only. No unnecessary limitations are to be understood therefrom. The entire disclosure of any article, patent or patent application identified herein is hereby incorporated by reference.

What is claimed is:

1. A sizing device for measuring a valve annulus, said sizing device comprising:
an elongate segment having first and second ends;
a sizer attached to the first end of the elongate segment, wherein the sizer is shaped to match a native mitral valve annulus at a portion of a cardiac cycle and/or is shaped to match a saddle shape of a corresponding annuloplasty device.

2. A sizing device for measuring a valve annulus, said sizing device comprising:
an elongate segment having first and second ends;
a first sizer attached to the first end of the elongate segment; and
a second sizer attached to the second end of the elongate segment, wherein the first and second sizers have different shapes that correspond to the same size of valve annulus.

3. The sizing device of claim 2, wherein the first and second sizers differ in two-dimensional and/or three-dimensional shape.

4. The sizing device of claim 2, wherein the first sizer is planar in shape and the second sizer has three-dimensional shape.

5. The sizing device of claim 2, wherein the first and second sizers include opposing notches located on opposite sides of the sizers, wherein the opposing notches are provided to facilitate viewing of portions of the valve annulus upon placement of the sizers into close proximity to the valve annulus.

6. A method of measuring a valve annulus of a heart, the method comprising the steps of:
providing a plurality of sizing devices, each sizing device comprising:
an elongate segment having first and second ends;
a first sizer attached to the first end of the elongate segment; and
a second sizer attached to the second end of the elongate segment;
wherein the first and second sizers have different shapes that correspond to the same size of valve annulus;
selecting one of the plurality of sizing devices;
positioning the first sizer of the one sizing device on the valve annulus; and
turning the one sizing device such that the first sizer is moved off the valve annulus and such that the second sizer is positioned on the valve annulus.

7. The method of claim 6, further comprising the step of:
repeating the selecting, positioning and turning steps until the size of the valve annulus is determined.

8. The method of claim 7, wherein the plurality of sizing devices correspond to a plurality of annuloplasty devices, the method further comprising the steps of:
providing a plurality of annuloplasty devices; and
selecting one of said annuloplasty devices which has the size that corresponds to the determined size of the valve annulus.

9. The method of claim 8, further comprising the step of:
implanting the selected one annuloplasty device on the valve annulus.

10. The method of claim 6, wherein the first and second sizers differ in two-dimensional and/or three-dimensional shape.

11. The method of claim 6, wherein the first sizer is planar in shape and the second sizer has three-dimensional shape.

12. The method of claim 6, wherein the first and second sizers include opposing notches located on opposite sides of the sizers, wherein the opposing notches are provided to facilitate viewing of portions of the valve annulus upon placement of the sizers into close proximity to the valve annulus.

13. A system for measuring a valve annulus of a heart, the system comprising:
a plurality of sizing devices each sizing device comprising:
an elongate segment having first and second ends;
a first sizer attached to the first end of the elongate segment; and
a second sizer attached to the second end of the elongate segment;
wherein the first and second sizers have different shapes that correspond to the same size of valve annulus;
wherein each of the plurality of sizing devices corresponds to one of a plurality of valve annulus sizes that also corresponds to one of a plurality of annuloplasty devices.

14. The system of claim 13, wherein the first and second sizers differ in two-dimensional and/or three-dimensional shape.

15. The system of claim 13, wherein the first sizer is planar in shape and the second sizer has three-dimensional shape.

16. The system of claim 13, wherein the first and second sizers include opposing notches located on opposite sides of the sizers, wherein the opposing notches are provided to facilitate viewing of portions of the valve annulus upon placement of the sizers into close proximity to the valve annulus.
